(19) United States
(12) Plant Patent Application Publication
(10) Pub. No.: US 2006/0037115 P1
(43) Pub. Date: Feb. 16, 2006

(54) Almond tree named 'Folsom'

(75) Inventor: Albert Edwin Folsom, Clovis, CA (US)

Correspondence Address: Attn: Dennis Tarry;Dave Wilson Nursery, Inc., 19701 Lake Rd, Hickman, CA, 95323, US

(73) Assignee: Dave Wilson Nursery, Hickman, CA (US)

(21) Appl. No.: 10/918,859

(22) Filed: Aug. 16, 2004

(51) Int. Cl. A01H 5/00

(52) U.S. Cl. PLT/155

(57) ABSTRACT

An almond tree (Prunus, Dulcis) named ‘Folsom’ that blooms and ripens in the early season and produces a high quality, marketable crop.

The present invention relates to a new and distinct variety of almond tree that has been given the name ‘Folsom’.

The original plant was discovered in the early 1960's as a mature tree growing in an orchard of ‘Nonpareil’ (non-patented) and ‘Thompson’ (U.S. Plant Pat. No. 1,526) almond trees located on the N. W. comer of Millbrook and Alluvial Avenues in Fresno, Calif. The inventor noted that the Nonpareil trees developed symptoms of “non-infectious bud failure” while a particular tree, the Folsom tree, remained productive and did not exhibit symptoms of the disorder. It was further noted that the Folsom tree bloomed and harvested at the same time as the Nonpareil and exhibited similar nut quality and characteristics.

Nonpareil is one of the most desirable cultivators grown because of its excellent quality and earliness of harvest. The Nonpareil also requires cross-pollination because it is not self-fertile. A variety that exhibits similar characteristics in bloom, nut quality, and harvest and can pollinate the Nonpareil would be desirable to plant The Folsom almond has exhibited these characteristics and under controlled conditions has been shown to pollinate the Nonpareil.

### Asexual Reproduction

The first asexual reproduction of the new variety occurred in the late 1980's when wood was collected from the original tree and grafted onto an existing tree in the inventors orchard located at 8510 N. Chestnut Ave., Clovis, Calif. Three additional trees were grafted on the inventors property in the late 1990's.

Budwood, from these trees, was collected in 2001 by Dave Wilson Nursery, and budded onto one each of ‘Nemaguard’ (non-patented) and ‘Atlas’ (U.S. Plant Pat. No. 8,913) rootstock located at 21200 E. Dinuba Ave., Reedley, Calif.

All asexually reproduced trees exhibit similar characteristics as described herein and are similar to those characteristics observed by the inventor on the original Folsom tree.

### SUMMARY

The Folsom almond is characterized principally to novelty by producing a consistently high quality nut similar to the Nonpareil variety. The present cultivator blooms with and is harvested at the same time as the Nonpareil.

Further, tree vigor, growth habit, and productivity appear to be similar to Nonpareil. Under controlled conditions, Folsom has been shown to pollinate the self-infertile variety Nonpareil, which is a desirable characteristic, and which needs to be further investigated in field trials.

### DESCRIPTION OF DRAWINGS

FIG. 1 is a picture of Folsom tree.

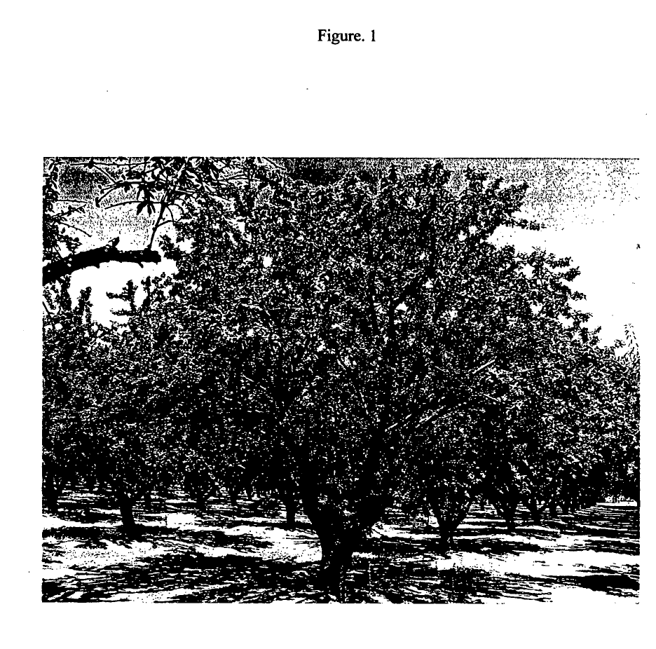

FIG. 2 is a picture of nuts and foliage.

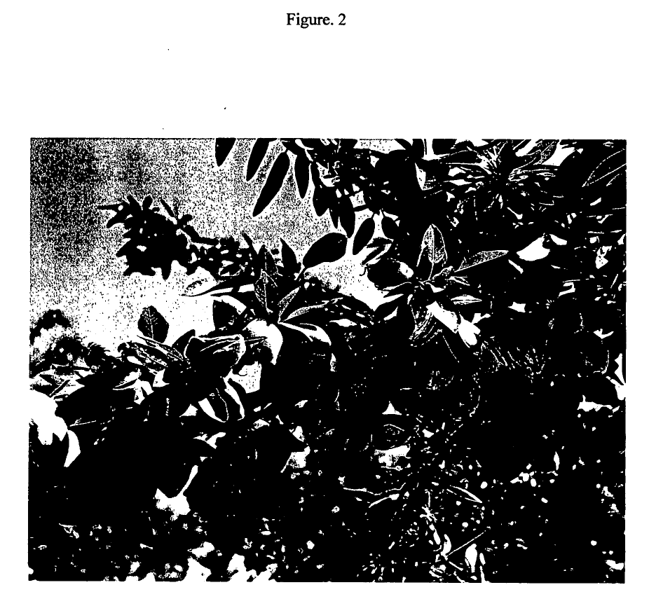

FIG. 3 is a picture of flower.

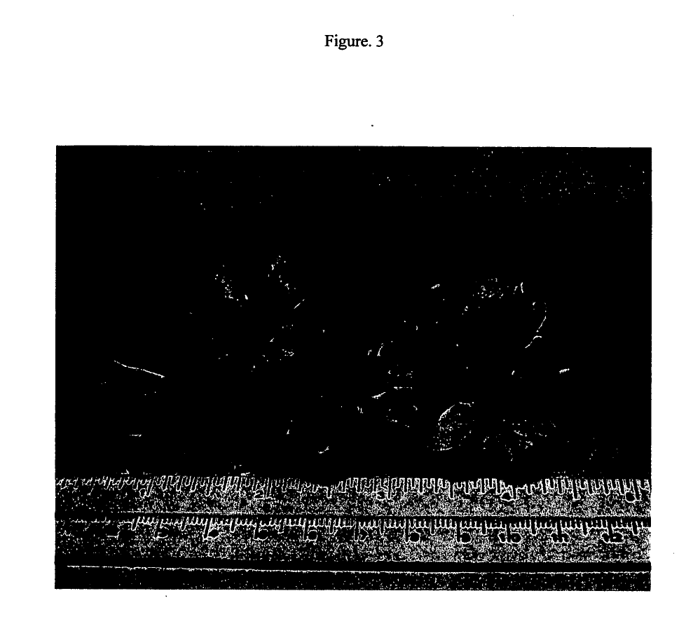

FIG. 4 is a picture of inuature nuts.

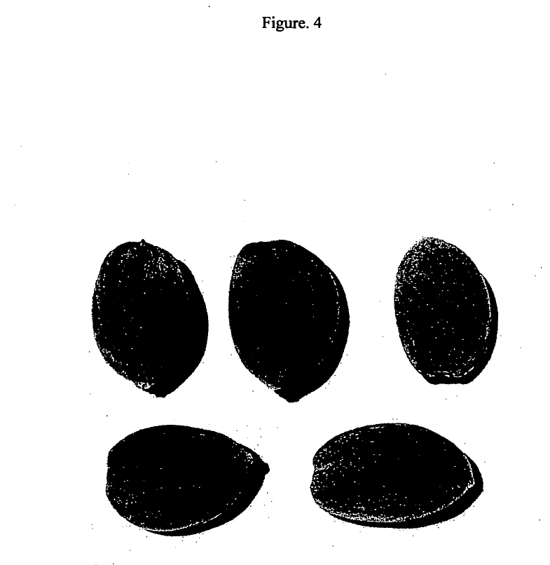

FIG. 5 is a picture of mature nuts.

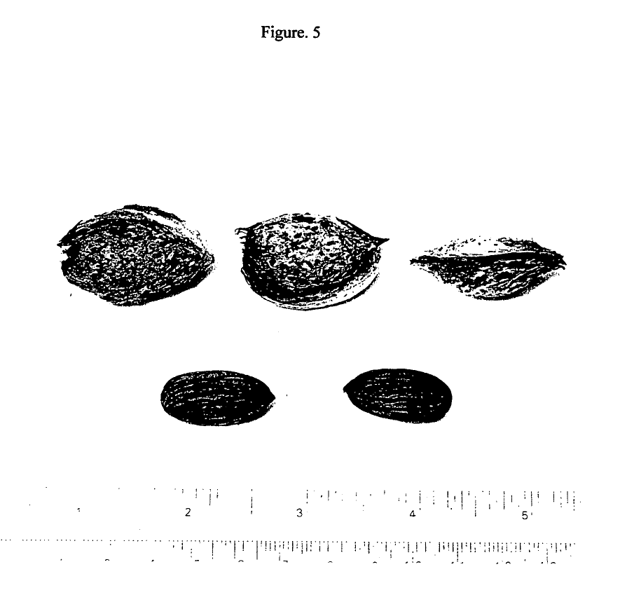

### DETAILED DESCRIPTION OF VARIETY

Specific botanical details of this new and distinct variety of almond have been observed under the ecological conditions prevailing at the inventors orchard located at 8510 N. Chestnut Ave., Clovis, Calif. and Dave Wilson Nursery located at 21200 E. Dinuba Ave., Reedley, Calif.

All major color code designations are referenced by The Royal Horticultural Society (RHS) Colour Chart, 3^{rd} edition, 1995. Botanical nomenclature is referenced by Harrington, H. D. and L. W. Durrell 1957, How to Identify Plants, The Swallow Press Incorporated. 203 pp.
Tree: Generally.
   Size.—Medium.
   Vigor.—Medium.
   Density.—Medium.
   Figure.—Spreading. Primary limb angles approximately 40-45°, dependent on pruning practices.
   Bearing.—Regular.
   Production.—Appears to be similar to Nonpareil almond.
   Chilling.—Grown under typical Central California climatic conditions.
Trunk: Generally.
   Form.—Medium as compared to other almond cultivators.
   Bark.—Texture, rough.
   Bark color.—Brown R.H.S. 200A with grey cast
Branches: Generally.
   Form.—Medium as compared to other almond cultivators.
   Texture.—Bark smooth on 1-4 year old wood. Rough on wood 5 years and older
   Lenticils.—Numerous, small to medium in size.
   Color.—1 year old, brown R.H.S. 200D with grey cast, sometimes interspersed with medium green R.H.S. 145A. 2 year old, grey green R.H.S. 197A to R.H.S. 197B.
Shoots: Generally.
   Size.—Medium, variable from 6 to 65 cm in length.
   Form.—Straight, generally lacking current year laterals.
   Texture.—Smooth.
   Color.—Medium green R.H.S. 145A. Generally lacking anthocyanin.
   Leaf buds.—Terminal, short and pointed. Lateral, small and triangular. Length and width approximately 2 to 4 cm.
   Color.—Dark brown, darker than R.H.S. 200A.
   Flower buds.—Conical in shape, 2 to 3 times larger than leaf buds.
   Color.—Dark browvn R.H.S. 200A to R.H.S. 200B.
   Position.—Lateral, 0 to 4 flower buds per node.
   Nodes.—5 to 35 in number. Intemodal spacing approximately 1 to 2.5 cm.
Spurs: Generally.
   Position.—Usually occur on older wood.
   Size.—Thickness, 0.3 to 0.9 cm. Length, 1 to 3.9 cm.
   Color.—Brown, R.H.S. 200A with grey cast.
   Viability.—May remain viable from 1 to 4 years producing both leaf and from 0 to 5 flower buds. Occasionally, short shoots may develop from a spur.
Leaves: Generally.
   Size.—Medium, average length, 8.5 cm. Average width, 19 cm. Ratio of blade to length, approximately 0.31.
   Shape.—Long, elliptical, tapering to apex.
   Tip.—Acuminate, tapering to a short, abrupt tip.
   Base.—Rounded to slightly oblique.
   Thickness.—Medium.
   Texture.—Smooth.
   Margin.—Crenate.
   Blade Color.—Upper surface R.H.S. 146A Lower surface RH.S. 147B.
   Midrib.—Distinct, upper surface color R.H.S. 146D. Lower surface color R.H.S. 145C.
   Leaf glands.—Inconspicuous, globose, generally 0 to 6 present, alternate on petiole, occasionally occurring on leaf base.
   Petiole.—Medium length and thickness with distinct groove on upper surface. Ratio of petiole length to leaf length approximately 0.28.
   Color.—Same as midrib.
   Stipules.—Absent.
Inflorescence: Generally.
   Bloom.—10% on Feb. 22, 2004 — Full on Feb. 27, 2004 at Dave Wilson Nursery, Reedley, Calif.
   Flower.—Complete and perfect. Stamen and petal insertion, perigynous. Ovary, superior. Flower parts considered regular.
   Petals.—Distinct, 5 to 6 present. Petals may overlap near their median margins when fully open.
   Petal shape.—Broadly ovate, apex distinctly retuse. Base tapering to a short, abrupt claw.
   Petal length.—1.5 to 2.0 cm.
   Petal width.—1.3 to 1.4 cm.
   Color.—Pink bud, apex is R.H.S. 65B. Full bloom, showy, pale white, lighter than R.H.S. 62D.
   Flower size.—Medium, averaging 3.3 cm in diameter.
   Sepals.—5-6 present, length 0.8 to 1.2 cm.
   Sepal color.—Varies from yellow-green R.H.S. 144B to greyed- purple R.H.S. 183C. A distinct midline R.H.S. 183C often present.
   Stamnens.—14 to 30 present Length varies, but does not project beyond petals. Anthers versatile, filaments white, lacking pigment.
   Pistil.—1, rarely 2 present. Length, 1 to 1.7 cm, generally straight and as long or longer than stamens. Pistil normally included in corolla, but may occasionally be exserted. Ovary and lower portion of style pubescent, upper portion of style and stigma lacking pubescence.
Immature fruit: Generally.
   Hull color.—Grey Green R.H.S. 195A.
   Pubescence.—Fine whitish grey evenly distributed over the hull surface.
   Form.—Length to width, ovate, tapering to a blunt point at apex.
   Base.—Straight.
   Apical view.—Generally fusiform in shape.
   Ventral edge.—Broadly rounded with a shallow but distinct suture line.
   Dorsal edge.—Broadly curved from base to tip, being gently tapered at base and more rounded towards tip.
   Apical tip.—Generally oblique to the fruit axis. Style rudiments often present.
   Hull texture.—Short basal grooves present. Ventral suture line located on prominent ridge. A less distinct dorsal ridge is occasionally present. The remainder of the fruit surface is smooth and even.
   Peduncle attachment.—Firmly attached to fruit, medium in size and round to oval in shape. Distinct depression observed when peduncle is separated from fruit.
Mature fruit: Generally.
   Dehiscence.—Occurs along the ventral suture line. The nut separates easily from the hull frequently leaving the mesocarp attached to the hull.
   Nut size.—Average length, 2.9 cm. Width 1.8 cm. Thickness, medium.
   Shape.—Ovate.
   Apex.—Acute, generally oblique to the axis.
   Shell.—Soft, often split along ventral edge. Pores poorly developed, few in number.
   Base.—Square to ventrally oblique.
   Peduncular scar.—Small, inconspicuous.
   Wing.—Broad and thin.
   Color.—Outer surface, R.H.S. 165C to R.H.S. 167D. Inner surface, R.H.S. 167D.
   Kernel size.—Average length, 2.1 cm. Width, 1.2 cm. Thickness, medium.
   Shape.—Ovate.
   Apex.—Acute, oblique to the axis.
   Base.—Generally truncate, rounded on the ventral edge.
   Hilum.—Ovate.
   Pellicle.— Thin, color R.H.S. 164C.
Percent of kernel to nut, approximately 63%. Number of doubles is low.
   Flavor.—Sweet.
   Quality.—Good.
   Disease resistance.—Appears to be similar to other almond cultivators grown under central valley growing conditions.
   Insect resistance.—Susceptibility likely similar to other soft- shell varieties, such as Nonpareil, grown in California.

Although this new variety of almond tree possesses the described characteristics as noted above, variations of those characteristics may occur due to changes in growing location and conditions, fertilization, and other cultural practices.

We claim:

1. A new and distinct variety of almond tree as illustrated and described herein, which is characterized as to novelty by its ability to bloom and harvest in the early season.
